(19) United States
(12) Patent Application Publication
(10) Pub. No.: US 2018/0269510 A1
(43) Pub. Date: Sep. 20, 2018

(54) BIOSENSORS WITH A DIRECT ELECTRICAL OUTPUT

(71) Applicant: University of Massachusetts, Boston, MA (US)

(72) Inventors: Derek R. Lovley, Amherst, MA (US); Toshiyuki Ueki, Amherst, MA (US); Kelly P. Nevin, Amherst, MA (US)

(21) Appl. No.: 15/609,036

(22) Filed: May 31, 2017

Related U.S. Application Data
(63) Continuation-in-part of application No. PCT/US15/62994, filed on Nov. 30, 2015.
(60) Provisional application No. 62/086,148, filed on Dec. 1, 2014.

(51) Int. Cl. H01M 8/16; C12N 1/20; C12N 15/72; C12N 15/63; C12N 15/52; C12N 15/01

(52) U.S. Cl. CPC H01M 8/16; C12N 1/20; C12N 15/72; C12Y 203/03001; C12N 15/52; C12N 15/01; C12N 15/635

(57) ABSTRACT

A biosensor that employs a bacterium in a biofilm present on one of two electrodes of an electrochemical cell. The bacterium is genetically modified by having deleted at least one native gene that encodes for the enzymatic transformation of a molecular moiety, and having substituted for the deleted native gene a different gene having a transcription factor that is under the control of an inducible promoter in conjunction with an inducer molecule. When the molecular moiety is present in a specimen of interest that is in contact with the biofilm, an electric current is generated in response to the presence of the inducer molecule. In the absence of the inducer molecule, no electricity is generated. The electric signal that is generated can be analyzed to determine the presence and the quantity of the inducer molecule in the specimen of interest.

### CROSS-REFERENCE TO RELATED APPLICATIONS

This application is a continuation-in-part of co-pending International Patent Application No. PCT/US15/62994 filed Nov. 30, 2015, which application claims priority to and the benefit of then co-pending U.S. provisional patent application Ser. No. 62/086,148, filed Dec. 1, 2014, each of which applications is incorporated herein by reference in its entirety.

### STATEMENT REGARDING FEDERALLY FUNDED RESEARCH OR DEVELOPMENT

This invention was made with government support under Award Number DE-SC0006790 awarded by Department of Energy Subsurface Biogeosciences Research Program. The government has certain rights in the invention.

### FIELD OF THE INVENTION

The invention relates to biosensors in general and particularly to a biosensor that employs live microbes.

### BACKGROUND OF THE INVENTION

Biosensors are well known in the prior art. In general, biosensors operate by interacting with substances that one wishes to identify, which interaction generally results in a chemical change, such as a binding reaction, or a reaction that produces an identifiable chemical species.

The conventional methods for identifying whether a specific chemical reaction has occurred or a specific chemical product has been produced involve such techniques as direct chemical analysis, optical sensing of the activation or deactivation of a fluorescent marker, and measurement of spectra such as absorption spectra, Raman spectra, or the like, in the visible, infrared or ultraviolet regions of the electromagnetic spectrum,

These conventional methods can be cumbersome and time consuming, and can require specific equipment which is best operated in a laboratory environment. Even with the advent of chip based sensors (e.g., so-called “gene chips”), there is still the necessity to use sophisticated apparatus to actually measure the results obtained by applying a material of interest to the gene chip.

There is a need for improved systems and methods for detecting and quantifying the presence of specific substances in a specimen of interest.

### SUMMARY OF THE INVENTION

According to one aspect, the invention features a genetically modified bacterium, comprising a bacterium configured to change a chemical state of a molecular moiety according to a chemical half-reaction with the concomitant consumption or generation of electrons, the bacterium having at least one deleted native gene that encodes for the enzymatic transformation of the molecular moiety, and having substituted for the deleted native gene a different gene having a transcription factor that is under the control of an inducible promoter in conjunction with an inducer molecule.

In one embodiment, the bacterium is Geobacter sulfurreducens.

In another embodiment, the deleted gene encodes for the enzyme citrate synthase.

In yet another embodiment, the molecular moiety is acetate. In still another embodiment, the transcription factor is Lacl.

In a further embodiment, the inducer molecule is isopropyl β-D-1-thiogalactopyranoside (IPGT).

In yet a further embodiment, the transcription factor is TetR.

In an additional embodiment, the inducer molecule is anhydrotetracycline (AT).

According to another aspect, the invention relates to a method of detecting an inducer molecule. The method comprises the steps of providing a biofilm on one electrode of an electrochemical cell, the biofilm comprising a bacterium configured to change a chemical state of a molecular moiety according to a chemical half-reaction with the concomitant consumption or generation of electrons, the bacterium having deleted at least one native gene that encodes for the enzymatic transformation of the molecular moiety, and having substituted for the deleted native gene a different gene having a transcription factor that is under the control of an inducible promoter in conjunction with an inducer molecule; providing a specimen to be analyzed for the presence of the inducer molecule in contact with the biofilm; providing a quantity of the molecular moiety; sensing the presence of an electric current in the electrochemical cell; analyzing the electric current to determine whether the inducer molecule is present as a result; and performing at least one of recording the result, transmitting the result to a data handling system, or to displaying the result to a user.

In one embodiment, the bacterium is Geobacter sulfurreducens.

In another embodiment, the deleted gene encodes for the enzyme citrate synthase.

In yet another embodiment, the molecular moiety is a selected one of acetate.

In still another embodiment, the transcription factor is Lacl.

In a further embodiment, the inducer molecule is isopropyl β-D-1-thiogalactopyranoside (IPGT).

In yet a further embodiment, the transcription factor is TetR.

In an additional embodiment, the inducer molecule is anhydrotetracycline (AT).

In one more embodiment, the method further comprises the step of comparing the result against a reference value.

In still a further embodiment, the method further comprises the step of quantifying an amount of the inducer molecule.

The foregoing and other objects, aspects, features, and advantages of the invention will become more apparent from the following description and from the claims.

### BRIEF DESCRIPTION OF THE DRAWINGS

The objects and features of the invention can be better understood with reference to the drawings described below, and the claims. The drawings are not necessarily to scale, emphasis instead generally being placed upon illustrating the principles of the invention. In the drawings, like numerals are used to indicate like parts throughout the various views.

FIG. 1 is a schematic diagram that illustrates the time evolution of an electrical signal in a system or method according to principles of the invention.

FIG. 2 is a schematic of the TCA cycle in which the presence and participation of citrate synthase is highlighted by being identified with an oval.

FIG. 3A is a diagram that illustrates how one deletes the citrate synthase gene (gltA) to prevent expression of a functional citrate synthase in Geobacter sulfurreducens.

FIG. 3B is a diagram that illustrates how one adds a plasmid with gltA under the control of an inducible promoter in Geobacter sulfurreducens.

FIG. 3C illustrates a first embodiment, in which the Lacl/lPTG system is used in Geobacter sulfurreducens according to the principles of the invention.

FIG. 4A is a graph of the growth of a biofilm observed by measuring the optical density at 600 nm (OD₆₀₀) as a function of time with IPTG (+IPTG) and without IPTG (−IPTG).

FIG. 4B is a graph of the growth of a biofilm observed by measuring the optical density at 600 nm (OD₆₀₀) as a function of time with AT (+AT) and without AT (−AT).

FIG. 5A is a schematic diagram that illustrates the repression of transcription.

FIG. 5B is a schematic diagram that illustrates that when repression of transcription occurs as in FIG. 5A, no signal is observed, and the acetate electrochemical reduction half reaction does not proceed.

FIG. 6A is a schematic diagram that illustrates the derepression of transcription (e.g., transcription is signaled to occur).

FIG. 6B is a schematic diagram that illustrates that when transcription occurs as in FIG. 6A, an electrical signal is observed, and the acetate electrochemical reduction half reaction proceeds.

FIG. 7A is an image of the optical density as observed using a Western blot analysis as a function of time for a strain that was induced with IPTG.

FIG. 7B is a graph of the corresponding optical densities shown in FIG. 7A as determined with a densitometer vs. time for a strain that was induced with IPTG.

FIG. 8 is an image of a growth system showing an anode with a biofilm, a separator and a cathode.

FIG. 9 is a graph of electrical output of a biofilm of Geobacter sulfurreducens engineered with a chemical-sensing circuit having a graphite anode.

FIG. 10 is a graph of electrical output of a biofilm of Geobacter sulfurreducens engineered with a chemical-sensing circuit having a platinum wire anode.

### DETAILED DESCRIPTION

Biological sensors provide the possibility of sensing a wide diversity of molecules with high precision and low detection limits. We have developed and now describe a novel strategy for microbial sensors in which the sensors yield a direct electrical output. This contrasts with typical microbial-based sensors which produce a chemical reporter such as fluorescent protein.

In general terms, an inducer molecule is being detected by the sensing of a current generated by a redox reaction using a precursor molecule such as acetate, which molecule is present in excess in solution, and which molecule undergoes a transformation only when the inducer molecule activates a substituted gene. The transformation reaction only proceeds when the inducer molecule is present.

Sensors with a direct electrical output were devised and have been operated with the microorganism Geobacter sulfurreducens, exploiting its unique capability of transferring electrons to electrodes, thereby generating an electrical current.

Our systems and methods are unique because we do not control the expression of microbial components that form electrical contacts with electrodes. Rather, we initially establish a biofilm on the reporting electrode and then control current production by controlling whether the organism is capable of metabolizing an organic substrate that is the source of electrons for current generation.

We genetically engineered Geobacter sulfurreducens to delete its native gene for citrate synthase, a key enzyme for the anaerobic oxidation of acetate, and introduced another citrate synthase gene that was under the control of an inducible promoter. In one embodiment, the detection switch is based on the LacI/IPTG system for the control. The biofilm specifically generated high currents only when the IPTG inducer molecule was present.

In another embodiment, a sensor strain was generated with TetR/AT system. These results demonstrate that a wide diversity of sensor modules can readily be inserted to produce sensor for a broad range of chemicals.

The systems and methods of the invention provide a method for rapid and direct electrical output by a biological sensor.

In addition to biosensing, it is expected that these systems and methods can be adapted for biocomputing. Genetic circuits which perform various calculations within bacteria with a fluorescent protein output have been described. It is believed that these circuits could be modified to produce an electrical output using the systems and methods described here. The topic of biocomputing has been discussed in the published literature. See, for example, Yaakov Benenson, “Biomolecular computing systems: principles, progress and potential,” Nature Reviews Genetics, Volume 13, July 2012, pages 455-466, published online 12 Jun. 2012, which document is incorporated by reference herein in its entirety.

Geobacter sulfurreducens can be grown on anodes comprised of a diversity of conductive materials. In particular we have described the apparatus and methods for preparing and using biofilms of various organisms, including specifically Geobacter sulfurreducens, in the following published patent documents: Microbial Fuel Cells, U.S. Pat. No. 8,283,076 B2 to Lovley et al, issued Oct. 9, 2012, Aerobic Microbial Fuel Cell, U.S. Pat. No. 8,663,852 B2 to Nevin et al, issued Mar. 4, 2014, and Microbial Production Of Multi-Carbon Chemicals And Fuels From Water And Carbon Dioxide Using Electric Current, by Lovley et al., U.S. Patent Application Publication No. 2012/0288898 A1, published on Nov. 15, 2012, the disclosure of each of which is incorporated by reference herein in its entirety for all purposes.

In the present invention, a genetically modified form of Geobacter sulfurreducens produces an electrical signal when the chemical of interest is detected, rather than continuously making current as would be the case in a microbial fuel cell.

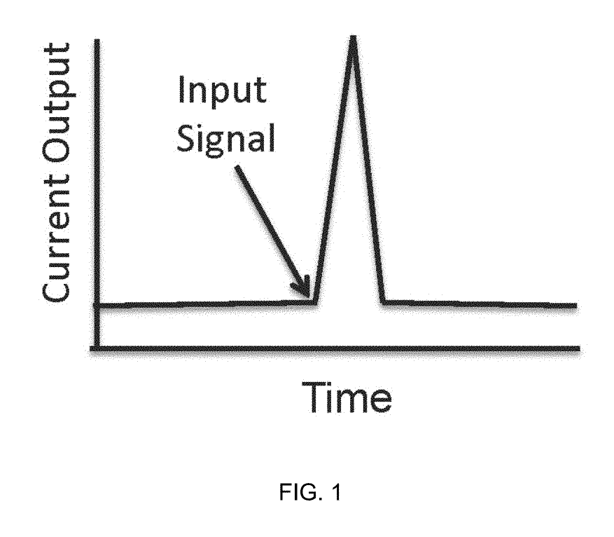

FIG. 1 is a schematic diagram that illustrates the time evolution of an electrical signal in a system or method according to principles of the invention. As seen in FIG. 1, no current is generated until there is provided a signal (the input signal) that turns on the detection system, which returns an electrical output signal when in the presence of the material that is specifically to be detected. The signal can be used to identify a presence of a material of interest, and/or, by measuring the height of the signal, quantifying the amount of the material of interest that is present. It is well known that extremely small numbers of electrons flowing in a circuit can be measured. If one knows how many electrons are produced or consumed in detecting one molecule of the substance of interest (e.g., an electrochemical half-reaction involving the material of interest is known), one can then measure the electrical current and determine quantitatively how much of that specific material is present. In some embodiments, it may be useful to have recorded signals from known concentrations of specific materials to use as reference signals for comparison. The reference signals can be recorded on a machine-readable medium.

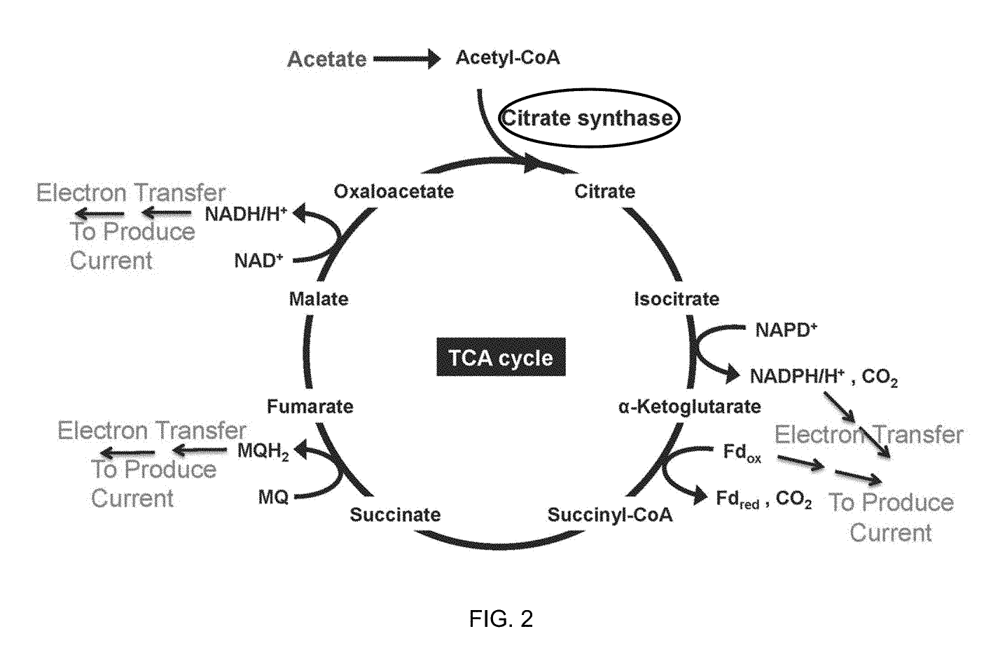

FIG. 2 is a schematic of the TCA cycle in which the presence and participation of citrate synthase is highlighted by being identified with an oval. As shown in FIG. 2, acetate is consumed in the TCA cycle and electrical current is produced at several points in the TCA cycle. In particular, Geobacter sulfurreducens extracts electrons for electric current production from acetate via the TCA cycle. In Geobacter sulfurreducens the gltA gene encodes citrate synthase, which is an enzyme that is required to introduce acetate into the TCA cycle. In the absence of citrate synthase, acetate cannot be converted to current by the TCA cycle.

Design of a Genetic Switch in Geobacter sulfurreducens

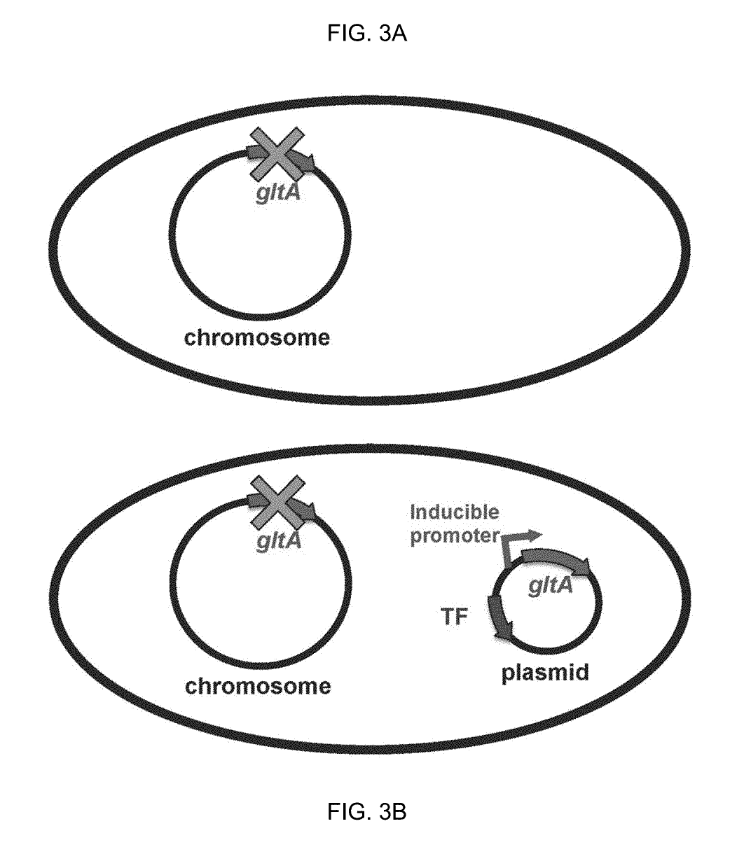

FIG. 3A and FIG. 3B illustrate how one can construct an inducible citrate synthase switch in Geobacter sulfurreducens in general.

In the method of constructing the inducible citrate synthase switch the first step is to delete the citrate synthase gene (gltA) to prevent expression of a functional citrate synthase.

FIG. 3A is a diagram that illustrates how one deletes the citrate synthase gene (gltA) to prevent expression of a functional citrate synthase in Geobacter sulfurreducens.

In the method of constructing the inducible citrate synthase switch the second step is to add a plasmid with gltA under the control of an inducible promoter.

FIG. 3B is a diagram that illustrates how one adds a plasmid with gltA under the control of an inducible promoter in Geobacter sulfurreducens.

FIG. 3C illustrates a first embodiment, in which the Lacl/lPTG system is used in Geobacter sulfurreducens according to the principles of the invention.

### METHOD OF USE

The method of use of the genetically modified biosensor, such as Geobacter sulfurreducens modified as described herein, is to grow a film of the microbe as described in the patent documents which have been incorporated herein by reference, and then to use the biofilm with acetate as the electron donor with citrate synthase expression under the control of an inducible promoter.

The inducible promoter is turned on by providing a signal molecule or inducer molecule. In some embodiments, a solution containing the inducer molecule is injected into the chamber containing the anode.

Table I presents examples of the material used to provide the “turn on” signal, and the corresponding transcription factor.

TABLE 1

| Signal | Transcription factor (TF) |
|---|---|
| IPTG | LacI |
| AT | TetR |

IPTG, Isopropyl β-D-1-thiogalactopyranoside
AT, Anhydrotetracycline

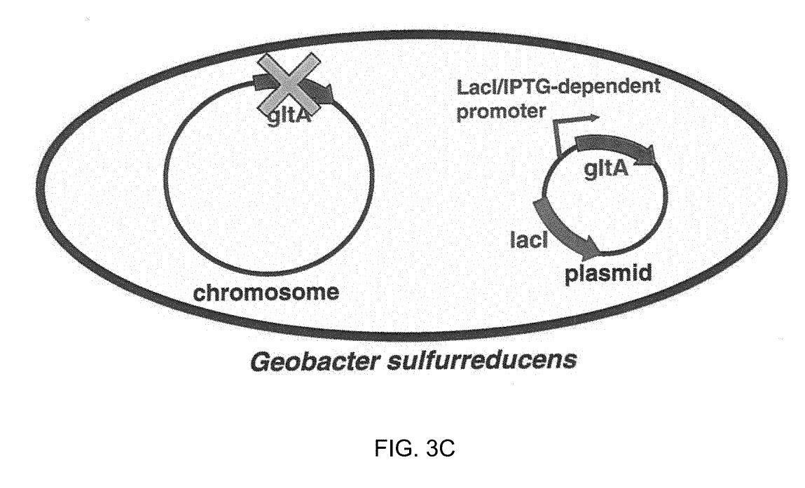

As illustrated in FIG. 4A and FIG. 4B, growth is observed only in the presence of the inducer molecule using Fumarate as the electron acceptor.

FIG. 4A is a graph of the growth of a biofilm observed by measuring the optical density at 600 nm (OD₆₀₀) as a function of time with IPTG (+IPTG) and without IPTG (−IPTG).

FIG. 4B is a graph of the growth of a biofilm observed by measuring the optical density at 600 nm (OD₆₀₀) as a function of time with AT (+AT) and without AT (−AT).

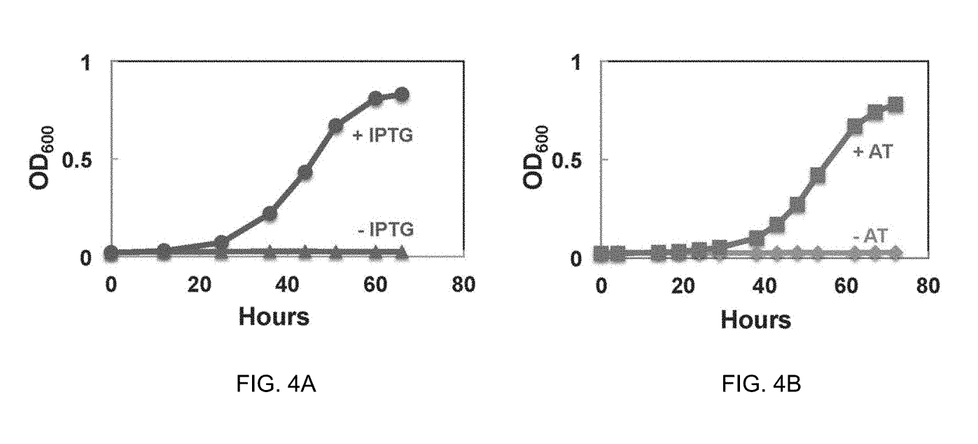

It is observed, that the current output substantially increases in response to an inducer molecule, and is absent in the absence of an inducer molecule. FIG. 5A and FIG. 5B illustrate that no electrical signal is generated by the oxidation of acetate when the promoter is turned off.

FIG. 5A is a schematic diagram that illustrates the repression of transcription.

FIG. 5B is a schematic diagram that illustrates that when repression of transcription occurs as in FIG. 5A, no signal is observed, and the acetate electrochemical reduction half reaction does not proceed.

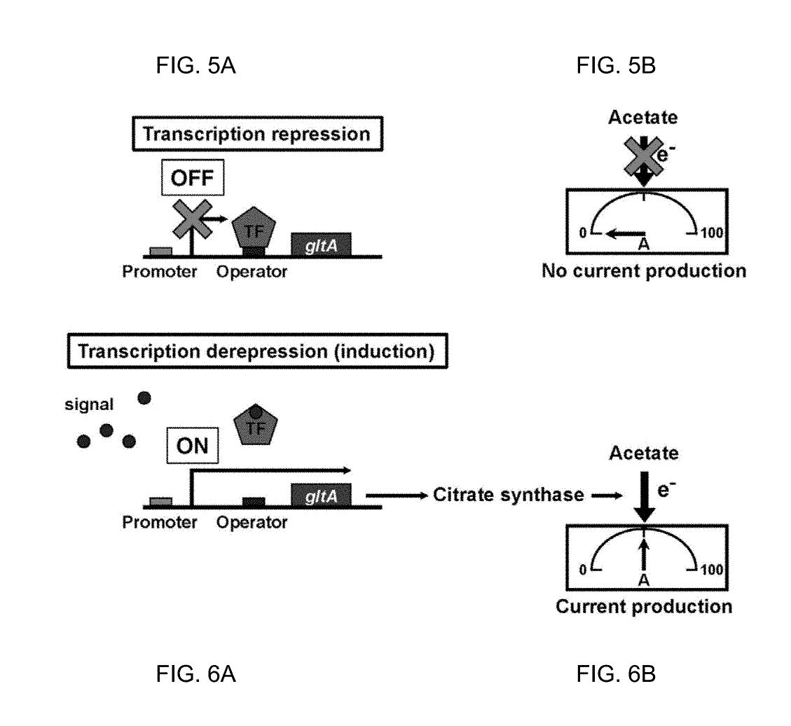

FIG. 6A is a schematic diagram that illustrates the derepression of transcription (e.g., transcription is signaled to occur).

FIG. 6B is a schematic diagram that illustrates that when transcription occurs as in FIG. 6A, an electrical signal is observed, and the acetate electrochemical reduction half reaction proceeds.

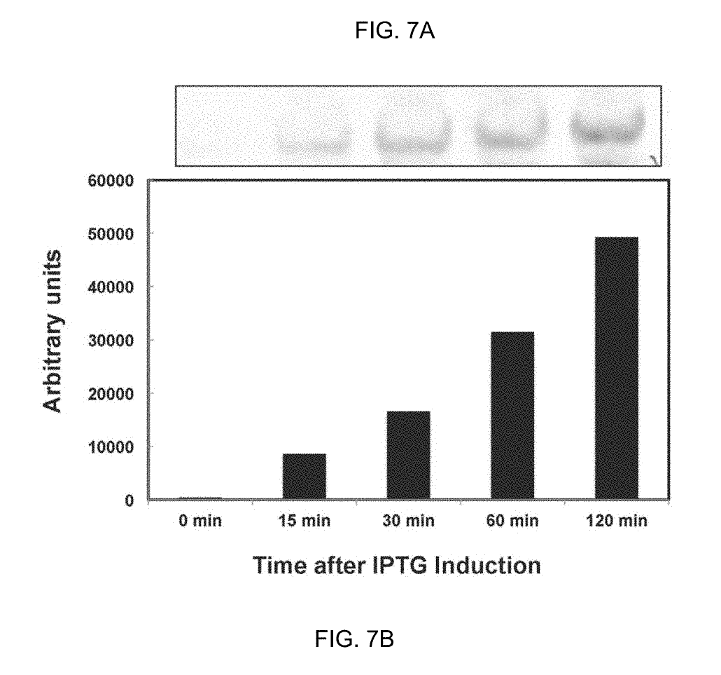

As illustrated in FIG. 7A and FIG. 7B, strains in which expression of citrate synthase was controlled with inducer molecules were successfully created.

FIG. 7A is an image of the optical density as observed using a Western blot analysis as a function of time for a strain that was induced with IPTG.

FIG. 7B is a graph of the corresponding optical densities shown in FIG. 7A as determined with a densitometer vs. time for a strain that was induced with IPTG.

Biofilms of G. sulfurreducens with citrate synthase under control of the IPTG-inducible promoter were grown on graphite anodes.

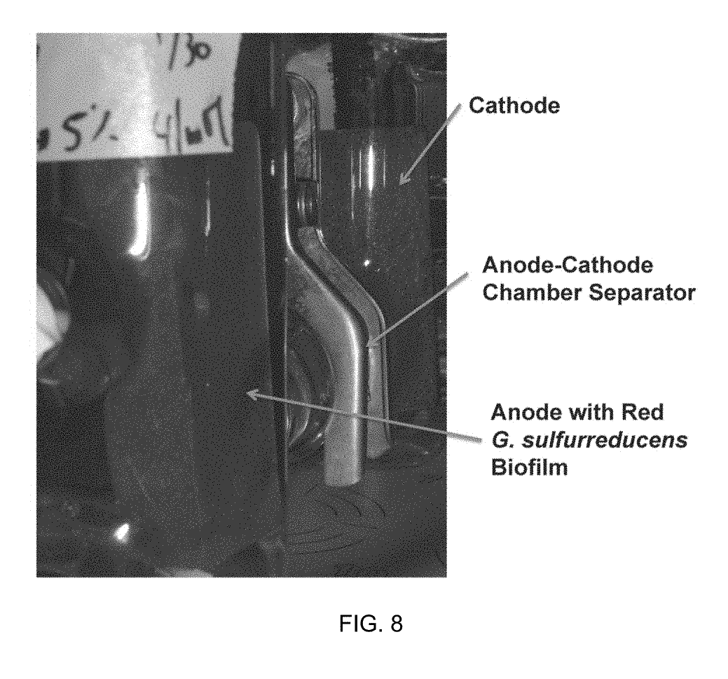

FIG. 8 is an image of a growth system showing an anode with a biofilm, a separator and a cathode.

A rapid current response is observed when ITPG is added to the graphite anode system.

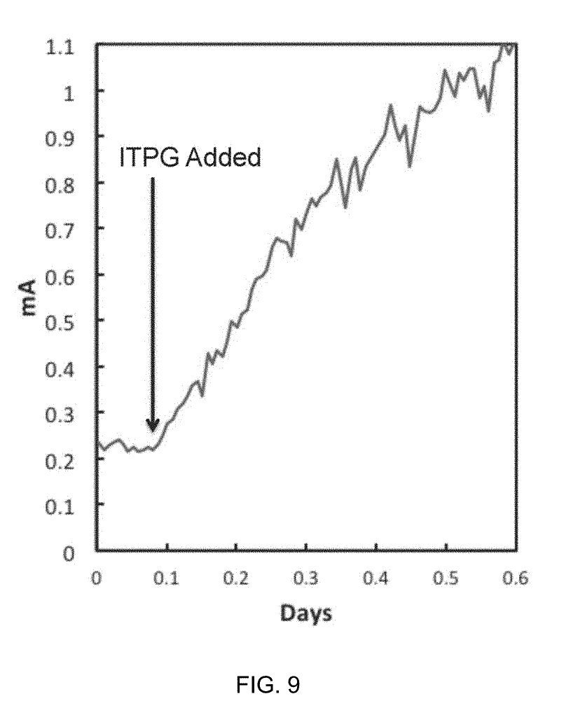

FIG. 9 is a graph of electrical output of a biofilm of Geobacter sulfurreducens engineered with a chemical-sensing circuit having a graphite anode.

A rapid current response is observed when ITPG is added to the platinum wire anode system.

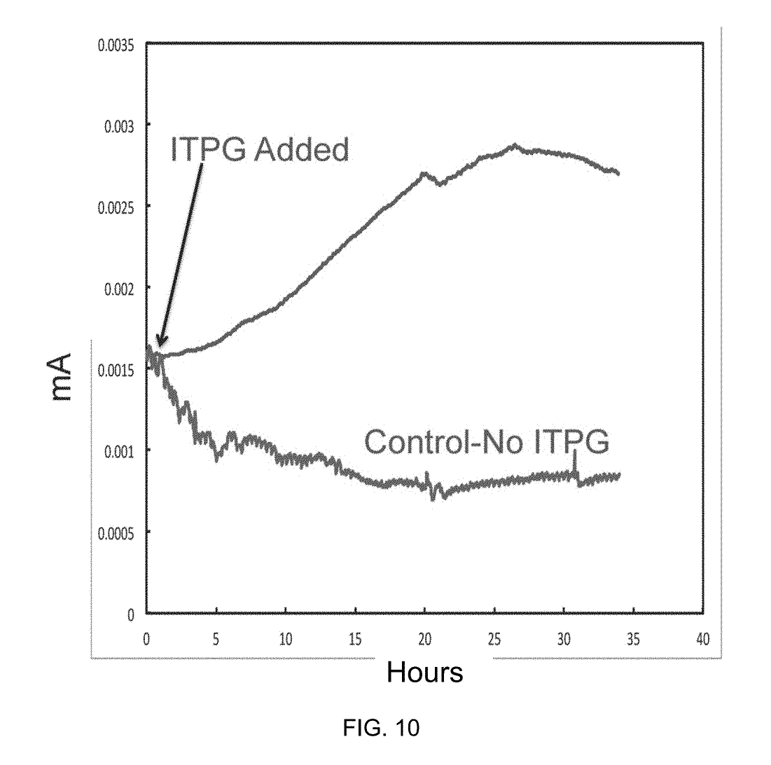

FIG. 10 is a graph of electrical output of a biofilm of Geobacter sulfurreducens engineered with a chemical-sensing circuit having a platinum wire anode.

### Additional Examples

It is believed that controlling a NADPH oxidoreductase in the same manner as citrate synthase will be effective.

### Growth of Biofilms

As discussed in U.S. Pat. No. 8,663,852 B2, G. sulfurreducens strain KN400 was inoculated 10% into an H-type fuel cell described in Bond, D. R. and D. R. Lovley (2003) “Electricity production by Geobacter sulfurreducens attached to electrodes” Appl. Environ. Microbiol. 69: 1548-1555 (hereinafter “Bond 2003”), with 40 mM fumarate and 10 mM acetate added. Biofims of KN400 were pregrown on anodes in H-type, two-chambered devices, in which the anode and cathode chambers are separated with a Nafion, cation-selective (or semi-permeable) membrane. The solid block graphite anodes that are typically employed were replaced with anodes with an interior chamber as shown in FIG. 2A and FIG. 2B of U.S. Pat. No. 8,663,852 B2. Anodes were poised at −400 versus Ag/AgCl with a potentiostat. Growth to an optical density at 600 nm, A600 nm, of 0.2 was followed by swapping the anode media to basal media with acetate as the electron donor and no soluble electron acceptor. Thus there was no electron acceptor other than the anode. Salt adapted G. sulfurreducens strain KN400 scraped from the starter cell, as described above, was directly inoculated into an H-type fuel cell containing marine media with 10 mM acetate added. In both cases the anodes were placed in flow through mode at 0.5 mL/min. Controls were the same configuration, except with solid block anodes and acetate remained in external media through the experiment.

When current production began fresh medium was continuously added to the anode chamber. Biofilms were grown until a current of 10 mA was achieved in the poised system. The medium input to the anode chamber was then changed to one in which the acetate was excluded and the internal chamber of the anode was filled with a concentrated (5 M) acetate solution. Current remained steady even though acetate became undetectable (<10 μM) in the external medium throughout the experiment.

### Definitions

Any reference in the claims to an electronic signal or an electromagnetic signal (or their equivalents) is to be understood that in a preferred embodiment the signal is a non-transitory electronic signal or a non-transitory electromagnetic signal. If the signal per se is not claimed, the reference may in some instances be to a description of a propagating or transitory electronic signal or electromagnetic signal.

Unless otherwise explicitly recited herein, any reference to “record” or “recording” is understood to refer to a non-volatile or non-transitory record or a non-volatile or non-transitory recording.

Recording the results from an operation or data acquisition, for example, recording results such as an electrical signal having a particular frequency or wavelength, or recording an image or a portion thereof, is understood to mean and is defined herein as writing output data in a non-volatile or non-transitory manner to a storage element, to a machine-readable storage medium, or to a storage device. Non-volatile or non-transitory machine-readable storage media that can be used in the invention include electronic, magnetic and/or optical storage media, such as magnetic floppy disks and hard disks; a DVD drive, a CD drive that in some embodiments can employ DVD disks, any of CD-ROM disks (i.e., read-only optical storage disks), CD-R disks (i.e., write-once, read-many optical storage disks), and CD-RW disks (i.e., rewriteable optical storage disks); and electronic storage media, such as RAM, ROM, EPROM, Compact Flash cards, PCMCIA cards, or alternatively SD or SDIO memory; and the electronic components (e.g., floppy disk drive, DVD drive, CD/CD-R/CD-RW drive, or Compact Flash/PCMCIA/SD adapter) that accommodate and read from and/or write to the storage media.

### THEORETICAL DISCUSSION

Although the theoretical description given herein is thought to be correct, the operation of the devices described and claimed herein does not depend upon the accuracy or validity of the theoretical description. That is, later theoretical developments that may explain the observed results on a basis different from the theory presented herein will not detract from the inventions described herein.

Any patent, patent application, patent application publication, journal article, book, published paper, or other publicly available material identified in the specification is hereby incorporated by reference herein in its entirety. Any material, or portion thereof, that is said to be incorporated by reference herein, but which conflicts with existing definitions, statements, or other disclosure material explicitly set forth herein is only incorporated to the extent that no conflict arises between that incorporated material and the present disclosure material. In the event of a conflict, the conflict is to be resolved in favor of the present disclosure as the preferred disclosure.

While the present invention has been particularly shown and described with reference to the preferred mode as illustrated in the drawing, it will be understood by one skilled in the art that various changes in detail may be affected therein without departing from the spirit and scope of the invention as defined by the claims.

What is claimed is:

1. A genetically modified bacterium, comprising:
a bacterium configured to change a chemical state of a molecular moiety according to a chemical half-reaction with the concomitant consumption or generation of electrons, said bacterium having deleted at least one native gene that encodes for the enzymatic transformation of said molecular moiety, and having substituted for said deleted native gene a different gene having a transcription factor that is under the control of an inducible promoter in conjunction with an inducer molecule.

2. The genetically modified bacterium of claim 1, wherein said bacterium is Geobacter sulfurreducens.

3. The genetically modified bacterium of claim 2, wherein said deleted gene encodes for the enzyme citrate synthase.

4. The genetically modified bacterium of claim 3, wherein said molecular moiety is a selected one of acetate and fumarate.

5. The genetically modified bacterium of claim 3, wherein said transcription factor is Lacl.

6. The genetically modified bacterium of claim 5, wherein said inducer molecule is isopropyl β-D-1-thiogalactopyranoside (IPGT).

7. The genetically modified bacterium of claim 3, wherein said transcription factor is TetR.

8. The genetically modified bacterium of claim 7, wherein said inducer molecule is anhydrotetracycline (AT).

9. A method of detecting an inducer molecule, comprising the steps of:
providing a biofilm on one electrode of an electrochemical cell, said biofilm comprising a bacterium configured to change a chemical state of a molecular moiety according to a chemical half-reaction with the concomitant consumption or generation of electrons, said bacterium having deleted at least one native gene that encodes for the enzymatic transformation of said molecular moiety, and having substituted for said deleted native gene a different gene having a transcription factor that is under the control of an inducible promoter in conjunction with an inducer molecule;
providing a specimen to be analyzed for the presence of said inducer molecule in contact with said biofilm;
providing a quantity of said molecular moiety;
sensing the presence of an electric current in said electrochemical cell;
analyzing said electric current to determine whether said inducer molecule is present as a result; and
performing at least one of recording said result, transmitting said result to a data handling system, or to displaying said result to a user.

10. The method of detecting an inducer molecule of claim 9, wherein said bacterium is Geobacter sulfurreducens.

11. The method of detecting an inducer molecule of claim 10, wherein said deleted gene encodes for the enzyme citrate synthase.

12. The method of detecting an inducer molecule of claim 11, wherein said molecular moiety is acetate

13. The method of detecting an inducer molecule of claim 11, wherein said transcription factor is Lacl.

14. The method of detecting an inducer molecule of claim 13, wherein said inducer molecule is isopropyl β-D-1-thiogalactopyranoside (IPGT).

15. The method of detecting an inducer molecule of claim 11, wherein said transcription factor is TetR.

16. The method of detecting an inducer molecule of claim 15, wherein said inducer molecule is anhydrotetracycline (AT).

17. The method of detecting an inducer molecule of claim 9, further comprising the step of comparing said result against a reference value.

18. The method of detecting an inducer molecule of claim 9, further comprising the step of quantifying an amount of said inducer molecule.

19. A method of making a biofilm that includes an inducer molecule, comprising the steps of:
providing a biofilm on one electrode of an electrochemical cell, said biofilm comprising a bacterium configured to change a chemical state of a molecular moiety according to a chemical half-reaction with the concomitant consumption or generation of electrons, said bacterium having deleted at least one native gene that encodes for the enzymatic transformation of said molecular moiety, and having substituted for said deleted native gene a different gene having a transcription factor that is under the control of an inducible promoter in conjunction with an inducer molecule by growth of said bacterium using one or more growth cycles.

20. The genetically modified bacterium of claim 19, wherein said bacterium is Geobacter sulfurreducens.
